(19) United States
(12) Patent Application Publication
(10) Pub. No.: US 2019/0161429 A1
(43) Pub. Date: May 30, 2019

(54) Aromatic Esters and Polyesters, Production Without Esterification Catalyst, and Use

(71) Applicant: ExxonMobil Chemical Patents Inc., Baytown, TX (US)

(72) Inventors: Mika L. Shiramizu, Houston, TX (US); Michael Salciccioli, Houston, TX (US); Neeraj Sangar, League City, TX (US); Ting Chen, Friendswood, TX (US)

(21) Appl. No.: 16/095,039

(22) PCT Filed: May 18, 2017
(86) PCT No.: PCT/US2017/033294

§ 371 Date: Oct. 19, 2018

Related U.S. Application Data
(60) Provisional application No. 62/353,335, filed on Jun. 22, 2016.

(30) Foreign Application Priority Data
Aug. 23, 2016 (EP) 16185357.7

(51) Int. Cl. C07C 67/08; C07C 67/03; C08G 63/185

(52) U.S. Cl. CPC C07C 67/08; C07C 2601/16; C08G 63/185; C07C 67/03

(57) ABSTRACT

This disclosure relates to methods for production of aromatic esters useful as plasticizers without using esterification catalyst, to the aromatic esters, and to polymer compositions containing the aromatic esters. It also relates to producing aromatic polyesters without using esterification catalyst. The aromatic esters and polyesters can be produced catalyst-free by esterifying carboxylic acids with alcohol(s) at high temperature and high pressure, namely at a temperature from 100° C. to 350° C. and a pressure ≥100 psig, preferably ≥600 psig. The aromatic esters and polyesters can also be produced by esterifying without esterification catalyst carboxylic acids with methyl or ethyl alcohol, separating the resulting methyl or ethyl esters from the carboxylic acid and any byproduct impurities, and then transesterifying with or without esterification catalyst the methyl or ethyl esters with alcohols and/or diols.

### PRIORITY CLAIM

This application claims priority to and the benefit of U.S. Ser. No. 62/353,335, filed Jun. 22, 2016 and EP 16185357.7, filed Aug. 23, 2016 and are incorporated by reference in their entirety.

### FIELD OF THE INVENTION

This disclosure relates to methods for production of aromatic esters useful as plasticizers without using esterification catalyst, to the aromatic esters, and to polymer compositions containing the aromatic esters, such as PVC. This disclosure also relates to methods for production of aromatic polyesters from aromatic methyl or ethyl esters produced without using esterification catalyst.

### BACKGROUND OF THE INVENTION

Plasticizers are incorporated into a resin (usually a plastic or elastomer) to increase the flexibility, workability, or distensibility of the resin. The largest use of plasticizers is in the production of “plasticized” or flexible polyvinyl chloride (PVC) products. Typical uses of plasticized PVC include films, sheets, tubing, coated fabrics, wire and cable insulation and jacketing, toys, flooring materials, such as, vinyl sheet flooring or vinyl floor tiles, adhesives, sealants, inks, and medical products such as blood bags and tubing, and the like.

Other polymer systems that use small amounts of plasticizers include polyvinyl butyral, acrylic polymers, nylon, polyolefins, polyurethanes, and certain fluoroplastics. Plasticizers can also be used with rubber (although often these materials fall under the definition of extenders for rubber rather than plasticizers). A listing of the major plasticizers and their compatibilities with different polymer systems is provided in “Plasticizers,” A. D. Godwin, in Applied Polymer Science 21st Century, edited by C. D. Craver and C. E. Carraher, Elsevier (2000); pp. 157-175.

Commonly assigned US 2015-0140350, incorporated by reference, identified plasticized vinyl chloride formulations using non-phthalate aromatic ester plasticizer. Commonly assigned US 2014-0315021, incorporated by reference, identified various blends of commercially available plasticizers with non-phthalate aromatic ester plasticizers. Co-pending U.S. Ser. No. 62/341,988, filed May 26, 2016, incorporated by reference, describes a process for producing purified aromatic esters.

Commonly assigned US 2015/0080545 and US 2015/0080546, incorporated by reference, describe aromatic polyesters.

Color body impurities make aromatic esters and polyesters less desirable for application, for example, aromatic esters having color and other impurities are less desirable as plasticizers. This imposes a significant hurdle for production of aromatic esters or polyesters because the color body impurity precursors could be present at an undetectable level for typical analysis methods, e.g., gas chromatography (GC) or high pressure liquid chromatography (HPLC). Even if measurement devices capable of detecting trace color body impurities are used, the purity specification for both feedstock and the produced aromatic esters may need to be set much higher to avoid color body impurities than to achieve other desired performance criteria resulting in added complexity and cost.

Therefore, a method for producing aromatic esters and polyesters that is more tolerant of color body impurity precursors and that proceeds at a commercially desirable reaction rate is desired.

### SUMMARY OF THE INVENTION

It is believed that production of the aromatic esters using esterification catalyst is one of the potential causes of color body impurity formation when color body impurity precursors are present, even in trace amounts. To address this issue, a method for producing the aromatic esters without catalyst has been developed.

The aromatic esters and polyesters can be produced catalyst-free by esterifying carboxylic acids with alcohol(s) at high temperature and high pressure, namely at a temperature from 100° C. to 350° C. and a pressure ≥100 psig, e.g., ≥250 psig, ≥500 psig, preferably ≥600 psig. Accordingly, in one aspect, the present invention relates to a method for producing aromatic esters comprises several steps. First, one or more feed compound(s) are provided of the following Formulas I(a) and/or I(b):

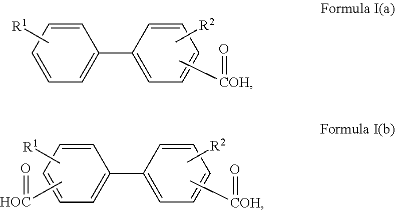

where R¹ is a hydrogen or an alkyl and R² is a hydrogen or an alkyl. Preferably, R¹ and/or R² is a methyl group. Preferable feed compounds are one or more isomers of methylbiphenylcarboxylic acid and/or one or more isomers of biphenyldicarboxylic acid. Second, the feed compound(s) are esterified without esterification catalyst with one or more C₁ to C₁₄ alcohols at a temperature 100° C. to 350° C. and a pressure ≥100 psig to form aromatic esters of the following Formulas III(a) and/or III(b):

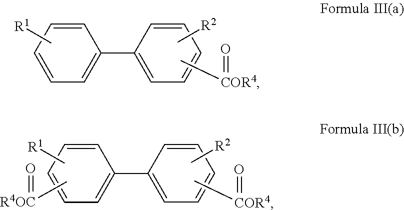

where R¹ and R² are as previously defined and R⁴ is an alkyl residual of the C₁ to C₁₄ alcohol(s). Preferably, the esterification is at a pressure of ≥250 psig, more preferably ≥500 psig, most preferably ≥600 psig. Preferably, R⁴ is an alkyl residual of C₁ to C₁₄ OXO-alcohol. In a more preferred embodiment, R⁴ is an alkyl residual of C₄ to C₁₄ alcohols.

In another aspect of the invention, a method for producing aromatic esters comprises several steps. First, provide one or more feed compound(s) (according to Formulas I(a) and I(b) as previously described) and one or more impurities. Second, esterify the feed compound(s) without esterification catalyst with methyl or ethyl alcohol (preferably methyl alcohol) at a temperature from 100° C. to 350° C. and a pressure ≥100 psig to form methyl or ethyl esters. Third, separate the methyl or ethyl esters from the one or more impurities. Fourth, transesterify the purified methyl or ethyl esters with C₄ to C₁₄ alcohols to form aromatic esters of C₄ to C₁₄ alcohols.

The invention also relates to producing aromatic polyesters from methyl or ethyl esters produced without esterification catalyst. The methyl or ethyl esters are transesterified with one or more diols and optionally dimethyl terephthalate with or without esterification catalyst at a temperature from 100° C. to 350° C. and a pressure at or above ambient pressure to form polyester. Preferably, the transesterification is with catalyst.

### DETAILED DESCRIPTION OF THE INVENTION

All numerical values within the detailed description and the claims herein are modified by “about” or “approximately” the indicated value, and take into account experimental error and variations that would be expected by a person having ordinary skill in the art. Unless otherwise indicated, room temperature is about 23° C.

A method for producing aromatic esters and polyesters without catalyst has been discovered to be more tolerant of color body impurity precursors and to proceed at a commercially desirable reaction rate.

“Esterifying” or “esterification” is reaction of a carboxylic acid moiety with an organic alcohol moiety to form an ester and water. The following equation depicts the esterification equilibrium reaction for the aromatic ester compounds described herein:

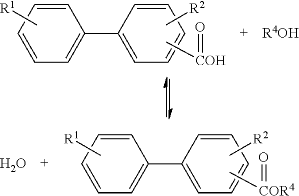

where R¹ and R² are as previously defined and R⁴ is a C₁ to C₁₄ alkyl group of the C₁ to C₁₄ alcohols (R⁴OH) and the aromatic esters (—COOR⁴).

Esterification conditions in the absence of any esterification catalyst can be a pressure above ambient pressure. In an embodiment of the invention, the esterification in the absence of esterification catalyst can be at a pressure ≥100 psig, ≥250 psig, or ≥500 psig, preferably ≥600 psig.

Esterification conditions in the absence of any esterification catalyst that produce commercially desirable reaction rates can be a temperature from 100° C. to 350° C., e.g., 150° C. to 300° C., 170° C. to 250° C., 180° C. to 210° C., 250° C. to 350° C. Above 350° C., undesirable reactions may occur such as decomposition. A low reaction temperature may result in an esterification rate that is commercially undesirable (i.e., too slow).

A method has been determined for catalyst-free production of aromatic esters of the above Formulas III(a) and III(b), where R¹ is a hydrogen or an alkyl, R² is a hydrogen or an alkyl, and R⁴ is an alkyl residual of C₁ to C₁₄ alcohol. In a preferable embodiment, R⁴ is an alkyl residual of C₄ to C₁₄ alcohol.

Aromatic esters can be produced by esterification of one or more feed compounds having the above Formulas I(a) and I(b) (reproduced here for convenient reference):

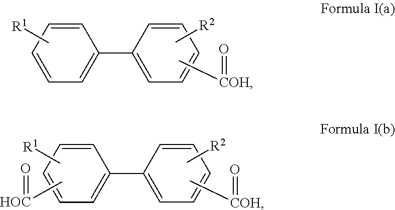

where R¹ is a hydrogen or an alkyl and R² is also a hydrogen or an alkyl. In any embodiment, the feed compounds comprise biphenyl substituted with one or more carboxylic acid groups. Optionally, the feed compounds comprise biphenyl substituted with one or more carboxylic acid groups and one or more additional alkyl groups. Preferably, the feed compounds comprise one or more isomers of methylbiphenylcarboxylic acid and/or biphenyldicarboxylic acid.

The feed compounds may be accompanied by various undesired impurities in the form of one or more of aldehydes, acetates, aldehyde-acids, acetate-acids, and color body impurity precursors. Some examples of impurity species include monoalcohols, monoacetates and monoaldehydes of the following formulas respectively:

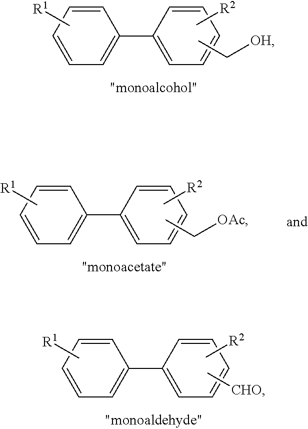

where R¹ and R² are as previously defined (R¹ is a hydrogen or an alkyl and R² is a hydrogen or an alkyl).

Additional impurity species that may be present include: acetooxymethylbiphenylcarboxylic acid, formylbiphenylcarboxylic acid, and biphenyldicarboxylic acid (when only mono-acid is desired) with the following formulas respectively:

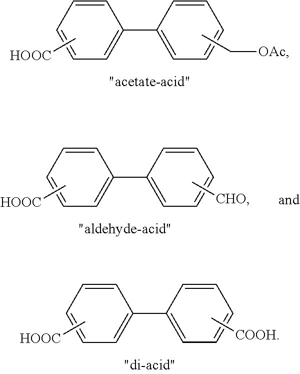

Of particular concern for production of the desired aromatic esters is the presence of color body impurities. It is believed that production of the aromatic esters using esterification catalyst is one of the potential causes of color body impurity formation when color body impurity precursors are present, even in trace amounts. Without being bound by any theory, some examples of color body impurities or precursors are believed to include fluorene, fluorenone, alkyl-substituted flourene, and alkyl-substituted fluorenone. It has been observed that esterification of analytically pure standard compounds of methylbiphenylcarboxylic acid with C₁ to C₁₄ alcohols in the presence of titanium (IV) esterification catalyst, even at relatively low reaction temperatures such as about 120° C., often formed some color in the aromatic esters. To address this issue, a method for producing the aromatic esters without catalyst has been developed.

In an embodiment, the feed compounds (according to Formulas I(a) and/or I(b) above) are esterified without esterification catalyst with one or more C₁ to C₁₄ alcohols at a temperature from 100° C. to 350° C. and a pressure ≥100 psig to form aromatic esters of the above Formulas III(a) and/or III(b) (reproduced here for convenience):

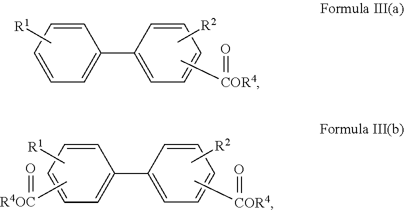

where R¹ is a hydrogen or an alkyl, R² is a hydrogen or an alkyl, and R⁴ is an alkyl residual of the C₁ to C₁₄ alcohol(s). Preferably, R⁴ is an alkyl residual of C₁ to C₁₄ OXO-alcohol. More preferably, R⁴ is an alkyl residual of C₄ to C₁₄ alcohols.
Aromatic Esters by Transesterification from Purified Esters of Methyl or Ethyl Alcohol

Co-pending U.S. Ser. No. 62/341,988, filed May 26, 2016, incorporated by reference here, describes a process for producing purified aromatic esters that involves esterifying the carboxylic acid feed compounds with methyl or ethyl alcohol, separating the resulting methyl or ethyl esters from impurities, and then transesterifying the separated (i.e., purified) methyl or ethyl esters with C₄ to C₁₄ alcohols to form C₄ to C₁₄ aromatic esters.

In an embodiment, a method for producing aromatic esters comprises providing one or more feed compound(s) (according to Formulas I(a) and I(b) as previously described) and one or more impurities; esterifying the feed compound(s) without esterification catalyst with methyl or ethyl alcohol (preferably methyl alcohol) at a temperature from 100° C. to 350° C. and a pressure ≥100 psig to form methyl or ethyl esters of previously described Formulas II(a) and/or II(b) (reproduced here for convenience):

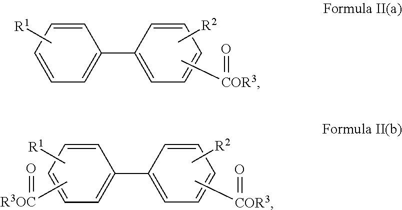

where R¹ and R² are as previously defined and R³ is an alkyl residual of the methyl or ethyl alcohol.

It has been discovered that the methyl or ethyl esters have a volatility that allows separation using commercially desirable amounts of heat and/or vacuum. The volatility of the methyl or ethyl esters is sufficiently different from the impurities to allow separation of the methyl or ethyl esters to a high purity. In an embodiment, the methyl or ethyl esters are separated to a purity of ≥99.0 wt % methyl or ethyl esters. Lower boiling impurities are more volatile than the methyl or ethyl esters. Higher boiling impurities are less volatile than the methyl or ethyl esters. In an embodiment, the methyl or ethyl esters are separated from substantially all the impurities to form purified methyl or ethyl esters.

The separation of methyl or ethyl esters can be by any known separation method utilizing relative volatility as the separation mechanism. The separation can be performed in one or more separation stages. In non-limiting embodiments, the separation of methyl or ethyl esters is by evaporative separation, flash separation, distillation, packed column, and/or vacuum distillation. In a preferred embodiment, the separation of methyl or ethyl esters is by vacuum distillation.

The purified methyl or ethyl esters are subsequently transesterified with C₄ to C₁₄ alcohol to form the desired C₄ to C₁₄ aromatic esters. The transesterification is performed with or without esterification catalyst, preferably with catalyst. “Transesterifying” or “transesterification” is an equilibrium reaction where the alkyl group R″ of an ester is exchanged with the alkyl group R′ of an alcohol. The following equation depicts the transesterification equilibrium reaction for the compounds described herein:

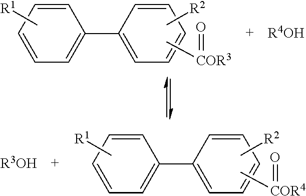

where R¹ and R² are as previously defined. R³ is the methyl or ethyl alkyl group of the methyl or ethyl esters, and R⁴ is the alkyl group of the C₄ to C₁₄ transesterification alcohols. Esters with C₄-C₁₄ alkyl groups can be produced in high purity from esters of methyl or ethyl alcohol by shifting equilibrium in favor of the C₄-C₁₄ alkyl group esters by i) heating a reaction mixture of methyl or ethyl ester and C₄-C₁₄ alcohol to evaporate the lower boiling methyl or ethyl alcohol as it is formed and/or ii) adding excess C₄-C₁₄ alcohol.

Transesterification conditions can be a pressure at or above ambient pressure.

Transesterification conditions that produce commercially desirable reaction rates can be a temperature from 100° C. to 350° C., e.g., 150° C. to 300° C., 170° C. to 250° C., 180° C. to 210° C., 250° C. to 350° C.

In embodiments of the invention, the esterification and/or transesterification is at a pressure of ≥100 psig, ≥250 psig, or ≥500 psig, preferably ≥600 psig.

In embodiments of the invention, the esterification and/or transesterification is at a temperature of ≥190° C., ≥195° C., or preferably ≥200° C.

In an embodiment, the purified methyl or ethyl esters are transesterified with or without esterification catalyst with C₄ to C₁₄ alcohol at a temperature from 100° C. to 350° C. and a pressure at or above ambient pressure to form aromatic esters of the previously defined Formulas III(a) and/or III(b) (reproduced for convenience):

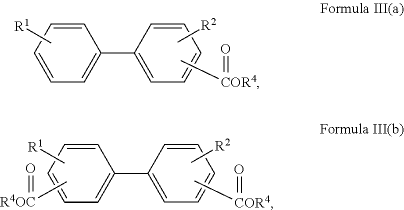

where R¹ is a hydrogen or an alkyl, R² is a hydrogen or an alkyl, and R⁴ is an alkyl residual of C₄ to C₁₄ alcohol.

In an embodiment, a method for producing aromatic esters comprises several steps. First, one or more feed compound(s) and one or more impurities are provided. The feed compound(s) have the following Formulas I(a) and/or I(b):

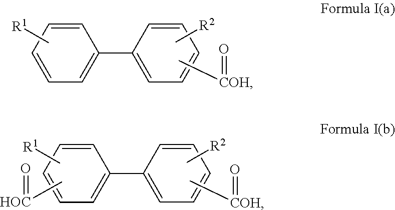

where R¹ is a hydrogen or an alkyl and R² is a hydrogen or an alkyl. The impurities comprise one or more of aldehydes, acetates, aldehyde-acids, acetate-acids, and color body impurity precursors. Preferably, R¹ and/or R² is a methyl group. Preferable feed compounds are one or more isomers of methylbiphenylcarboxylic acid and/or one or more isomers of biphenyldicarboxylic acid. Second, the feed compound(s) are esterified without esterification catalyst with methyl or ethyl alcohol (preferably methyl alcohol) at a temperature from 100° C. to 350° C. and a pressure ≥100 psig to form methyl or ethyl esters of the following Formulas II(a) and/or II(b):

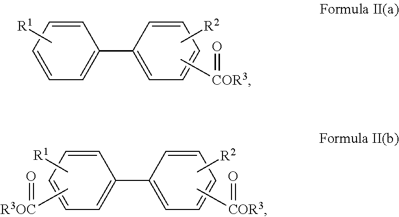

where R¹ and R² are as previously defined and R³ is an alkyl residual of the methyl or ethyl alcohol. Preferably, the esterification is at a pressure of ≥250 psig, more preferably ≥500 psig, most preferably ≥600 psig. Third, the methyl or ethyl esters are separated from substantially all the impurities to form purified methyl or ethyl esters. Fourth, the purified methyl or ethyl esters are transesterified with or without esterification catalyst with C₄ to C₁₄ alcohol at a temperature from 100° C. to 350° C. and a pressure at or above ambient pressure to form aromatic esters of the following Formulas III(a) and/or III(b):

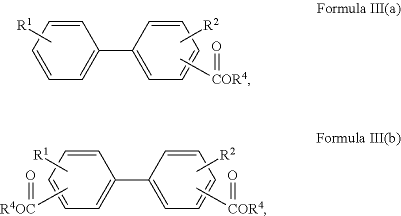

where R¹ and R² are as previously defined and R⁴ is an alkyl residual of the C₄ to C₁₄ alcohol(s). Preferably, the transesterification is with esterification catalyst.

In a preferred embodiment, the aromatic esters are methylbiphenylcarboxylic acid esters of C₄ to C₁₄ alcohols. Any known C₄ to C₁₄ alcohols can be used to form the aromatic esters by esterifying the feed compounds or transesterifying the purified methyl or ethyl esters. The desired aromatic esters can be formed with one or more C₄ to C₁₄ alcohols. In a preferred embodiment, the aromatic esters are formed by esterifying or transesterifying with C₄ to C₁₄ OXO-alcohols, which are isomeric linear, branched, or mixtures of linear and branched, organic alcohols.

OXO-alcohols can be prepared by hydroformylating olefins, followed by hydrogenation to form the alcohols. “Hydroformylating” or “hydroformylation” is the process of reacting a compound having at least one carbon-carbon double bond (an olefin) in an atmosphere of carbon monoxide and hydrogen over a cobalt or rhodium catalyst, which results in addition of at least one aldehyde moiety to the underlying compound. U.S. Pat. No. 6,482,972, which is incorporated herein by reference in its entirety, describes the hydroformylation (OXO) process. The resulting OXO-alcohols consist of multiple isomers of a given chain length due to the various isomeric olefins obtained in the oligomerization process, described below, in tandem with the multiple isomeric possibilities of the hydroformylation step.

Typically, the isomeric olefins are formed by light olefin oligomerization over heterogeneous acid catalysts, such as by propylene and/or butene oligomerization over solid phosphoric acid or zeolite catalysts. The light olefins are readily available from refinery processing operations. The reaction results in mixtures of longer-chain, branched olefins, which are subsequently formed into longer chain, branched alcohols, as described below and in U.S. Pat. No. 6,274,756, incorporated herein by reference in its entirety. Olefins for hydroformylation can also be prepared by dimerization of propylene or butenes through commercial processes such as the IFP Dimersol™ process or the Huls (Evonik) Octol™ process.

Branched aldehydes are then produced by hydroformylation of the isomeric olefins. The resulting branched aldehydes can then be recovered from the crude hydroformylation product stream by fractionation to remove unreacted olefins. These branched aldehydes can then be hydrogenated to form alcohols (OXO-alcohols). Single carbon number alcohols can be used in the esterification of the carboxylic acids described above, or differing carbon numbers can be used to optimize product cost and performance requirements. The “OXO” technology provides cost advantaged alcohols. Other options are considered, such as hydroformylation of C₄-olefins to C₅-aldehydes, followed by hydrogenation to C₅-alcohols, or aldehyde dimerization followed by hydrogenation to C₁₀ alcohols.

“Hydrogenating” or “hydrogenation” is addition of hydrogen (H₂) to a double-bonded functional site of a molecule, such as in the present case the addition of hydrogen to the aldehyde moieties of a di-aldehyde, to form the corresponding di-alcohol, and saturation of the double bonds in an aromatic ring. Conditions for hydrogenation of an aldehyde are well-known in the art and include, but are not limited to temperatures of 0-300° C., pressures of 1-500 atmospheres, and the presence of homogeneous or heterogeneous hydrogenation catalysts, such as, but not limited to Pt/C, Pt/Al₂O₃, Pd/Al₂O₃, and Ni. Useful hydrogenation catalysts include platinum, palladium, ruthenium, nickel, zinc, tin, cobalt, or a combination of these metals, with palladium being particularly advantageous.

Alternatively, the OXO-alcohols can be prepared by aldol condensation of shorter-chain aldehydes to form longer chain aldehydes, as described in U.S. Pat. No. 6,274,756, followed by hydrogenation to form the OXO-alcohols.

The C₄ to C₁₄ alcohols can be used individually or together in alcohol mixtures having different chain lengths, or in isomeric mixtures of the same carbon chain length to esterify the feed compounds or transesterify the purified methyl or ethyl esters and make aromatic esters of mixed alcohols for use as plasticizers. This mixing of carbon numbers and/or levels of branching in the alcohols can be advantageous to achieve the desired compatibility with PVC and to meet other plasticizer performance properties. The preferred alcohols for esterification or transesterification are those having from 4 to 14 carbons, more preferably C₅ to C₁₃ alcohols, still more preferably C₅ to C₁₁ alcohols, and even more preferably C₆ to C₁₀ alcohols.

In one embodiment, the preferred alcohols for esterification or transesterification are those which have an average branching of from 0.2 to 5.0 branches per molecule, and from 0.35 to 5.0 methyl branches per molecule, or even from 1.3 to 5.0 methyl branches per molecule. In a more preferred embodiment, the alcohols have from 0.05 to 0.4 branches per residue at the alcoholic beta carbon.

As a non-limiting example of suitable branched alcohols, the branching characteristics of OXO-alcohols are provided in Table 1, below.

TABLE 1
¹³C NMR Branching Characteristics of Typical OXO-Alcohols

| | | % of α- | | | Pendant | |
|---|---|---|---|---|---|---|
| OXO- | Avg. | Carbons w/ | β-Branches per | Total Methyls | Methyls per | Pendant Ethyls |
| Alcohol | Carbon No. | Branches^{a} | Molecule^{b} | per Molecule^{c} | Molecule^{d} | per Molecule |
| C₄^{e} | 4.0 | 0 | 0.35 | 1.35 | 0.35 | 0 |
| C₅^{f} | 5.0 | 0 | 0.30 | 1.35 | 0.35 | 0 |
| C₆ | — | — | — | — | — | — |
| C₇ | 7.2 | 0 | 0.13 | 2.2 | — | 0.04 |
| C₈ | 8.0 | 0 | 0.08 | 2.6 | — | — |
| C₉ | 9.3 | 0 | 0.09 | 3.1 | — | — |
| C₁₀ | 10.1 | 0 | 0.08 | 3.1 | — | — |
| C₁₂ | 11.8 | 0 | 0.09 | 3.9 | — | — |
| C₁₃ | 12.7 | 0 | 0.09 | 3.9 | — | — |

— Data not available.
^{a}—COH carbon.
^{b}Branches at the —CCH₂OH carbon.
^{c}This value counts all methyl groups, including C₁ branches, chain end methyls, and methyl endgroups on C₂+ branches.
^{d}C₁ branches only.
^{e}Calculated values based on an assumed molar isomeric distribution of 65% n-butanol and 35% isobutanol (2-methylpentanol).
^{f}Calculated values based on an assumed molar isomeric distribution of 65% n-pentanol, 30% 2-methylbutanol, and 5% 3-methylbutanol.

In a preferred embodiment of the invention, the esterification or transesterification alcohol (such as an OXO-alcohol) has 2.0 to 3.5 methyl branches per molecule, typically 2.1 to 3.3.

In general, for every polymer to be plasticized, a plasticizer is required with a good balance of polarity or solubility, volatility, and viscosity to have acceptable plasticizer compatibility with the resin. In particular, if the 20° C. kinematic viscosity is higher than 250 mm²/sec as measured by the appropriate ASTM test, or alternately if the 20° C. cone-and-plate viscosity is higher than 250 cP, this will affect the plasticizer processability during formulation, and can require heating the plasticizer to ensure good transfer during storage and mixing of the polymer and the plasticizer. Volatility is also an important factor which affects the aging or durability of the plasticized polymer. Highly volatile plasticizers will diffuse and evaporate from the plastic resin matrix, thus losing mechanical strength in applications requiring long term stability/flexibility. Relative plasticizer loss from a resin matrix due to plasticizer volatility can be roughly predicted by neat plasticizer weight loss at 220° C. using Thermogravimetric Analysis.

It was found that when C₄ to C₁₄ alcohols are used as reactants for the esterification or transesterification reactions described above, the resulting aromatic esters are in the form of relatively high-boiling liquids (having low volatility), which are readily incorporated into polymer formulations as plasticizers.

Any of the aromatic esters having alkyl residues of C₄ to C₁₄ alcohols, can be used as plasticizers for polymers, such as vinyl chloride resins, polyesters, polyurethanes, silylated polymers, polysulfides, acrylics, ethylene-vinyl acetate copolymer, rubbers, poly(meth)acrylics and combinations thereof, preferably polyvinylchloride.

### Aromatic Polyesters by Transesterification

Another aspect of the invention relates to producing aromatic polyesters from aromatic methyl or ethyl esters that are produced without esterification catalyst. Polyester may be produced by transesterifying diesters, e.g., diesters of methyl or ethyl alcohols although esters of C₁ to C₄ alcohols are also suitable for the invention, with diols and, optionally, dimethyl terephthalate. The diesters are reacted with diols to allow formation of polyester macromolecules.

Any of the methyl or ethyl diester isomers described herein are suitable as monomer for producing the polyesters by transesterification. Preferable methyl diesters include mixtures comprising at least 50 wt %, preferably from 90 to 99 wt %, of a compound of the formula:

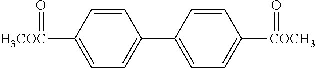

and at least 1 wt %, preferably from 1 to 10 wt %, of a compound of the formula:

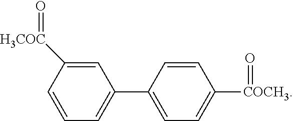

In the context of producing polyester, “transesterification” means exchange of an alkyl group R³ of a di-ester with the residual (R⁵OH) of a diol (di-alcohol) after one of the OH groups is separated. The following equation depicts the initiating transesterification polymerization equilibrium reaction for the compounds described herein:

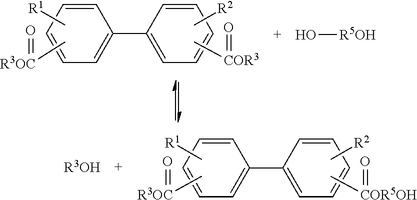

where R¹ and R² are as previously defined. R³ is a methyl or ethyl alkyl group of methyl or ethyl diesters, and HO—R⁵OH is a suitable diol as described below. Both the R³ and the R⁵OH of the equilibrium product depicted are available for further growth to form a polyester macromolecule. Growth of polyester macromolecule may be promoted by shifting equilibrium by removal of methanol (R³OH), e.g., by evaporation and/or by addition of excess diol.

In an embodiment of the invention, a method for producing polyester comprises: providing one or more feed compound(s) and one or more impurities, the feed compounds have the following Formula I(b):

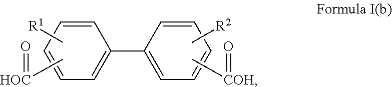

where R¹ is a hydrogen or an alkyl and R² is a hydrogen or an alkyl, the impurities comprise one or more of aldehydes, acetates, aldehyde-acids, acetate-acids, and color body impurity precursors; esterifying the feed compound(s) without esterification catalyst with methyl or ethyl alcohol (preferably methyl alcohol) at a temperature from 100° C. to 350° C. and a pressure ≥100 psig to form methyl or ethyl esters of the following Formula II(b):

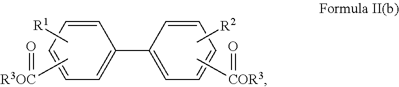

where R¹ and R² are as previously defined and R³ is an alkyl residual of the methyl or ethyl alcohol; separating the methyl or ethyl esters from substantially all the impurities to form purified methyl or ethyl esters; transesterifying the purified methyl or ethyl esters with one or more diols with or without esterification catalyst at a temperature from 100° C. to 350° C. and a pressure at or above ambient pressure to form polyester. Preferably, R¹ and/or R² is a hydrogen. Preferable feed compounds are one or more isomers of biphenyldicarboxylic acid. Preferably, the esterification is at a pressure of ≥100 psig, preferably ≥250 psig, more preferably ≥500 psig, most preferably ≥600 psig. Preferably, the transesterification is with catalyst.

Transesterification conditions that produce commercially desirable polyester reaction rates can be a temperature from 100° C. to 350° C., e.g., 150° C. to 300° C., 170° C. to 250° C., 180° C. to 210° C., 250° C. to 350° C.

Transesterification conditions can include a pressure at or above ambient pressure.

Generally, the transesterification reaction is conducted in the molten state and so the temperature is selected to be above the melting point of the monomer mixture but below the decomposition temperature of the polymer. The polyester may be first prepared in the molten state followed by a solid state polymerization to increase its molecular weight or intrinsic viscosity for applications like bottles.

Suitable diols for reaction with the above-mentioned diacid or diester compositions include alkanediols having 2 to 12 carbon atoms, such as monoethylene glycol, diethylene glycol, 1,3-propanediol, or 1,4-butane diol, 1,6-hexanediol, and 1,4-cyclohexanedimethanol.

Suitable diols used to form the polyesters (either by esterification or transesterification) have the following formulas:

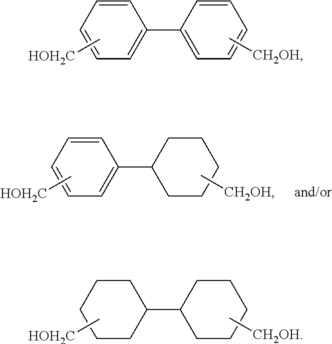

Preferable diols comprise a mixture of at least 50 wt %, preferably from 90 to 99 wt %, of one or more compounds having the formulas:

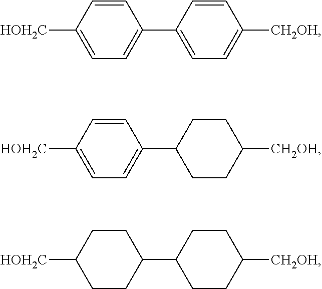 [SMILES: OCc1ccc(-c2ccc(CO)cc2)cc1 ; OCc1ccc(C2CCC(CO)CC2)cc1 ; OCC1CCC(C2CCC(CO)CC2)CC1]

and at least 1 wt %, preferably from 1 to 10 wt %, of one or more compounds having the formulas:

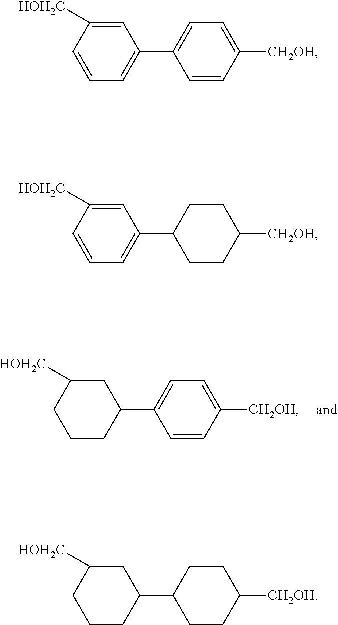 [SMILES: OCc1ccc(-c2cccc(CO)c2)cc1 ; OCc1cccc(C2CCC(CO)CC2)c1 ; OCc1ccc(C2CCCC(CO)C2)cc1 ; "and" ; OCC1CCC(C2CCCC(CO)C2)CC1]

### EXAMPLES

### Examples 1 Through 4

Procedure for Examples 1 to 4: A 300 or 600 mL Parr autoclave reactor was charged with carboxylic acid feed compounds and alcohol as specified in Table 2. The reactor was mounted with a condenser that was set to 2° C. The reactor was purged with nitrogen gas. After a pressure test to ensure seal, stirring was started at 600 rpm. The reactor was heated to either 200° C. or 250° C. as specified in Table 2. The reactor was pressurized and maintained at 600 psig via pressure regulator. Stirring was continued for 4 hours. Subsequently, heat was removed and pressure slowly reduced. Once reactor had returned to near ambient temperature, the reactor contents were collected and analyzed by gas chromatograph (GC).

TABLE 2
Examples 1-4 Esterification

| | Carboxylic Acid Feed | | Temp | Ester | |
|---|---|---|---|---|---|
| Example | Compound | Alcohol | ° C. | % Yield | Ester Produced |
| 1 | 3′-methyl-biphenyl-3- | 1-decanol | 250 | 100 | decyl 3′-methyl-biphenyl-3- |
| | carboxylic acid (5 g) | (122 g) | | | carboxylate |
| 2 | [1,1′-biphenyl]-4-carboxylic | methanol | 200 | 86 | methyl [1,1′-biphenyl]-4- |
| | acid (2.5 g) | (200 g) | | | carboxylate |
| 3 | 4′-methyl-[1,1′-biphenyl]-4- | methanol | 200 | 97 | methyl 4′-methyl-[1,1′- |
| | carboxylic acid (2.5 g) | (100 g) | | | biphenyl]-4-carboxylate |
| 4 | 3′-methyl-[1,1′-biphenyl]-3- | methanol | 200 | 98 | methyl 3′-methyl-[1,1′- |
| | carboxylic acid (2.5 g) | (100 g) | | | biphenyl]-3-carboxylate |

Regarding example 1, excess alcohol (1-decanol) and the decyl ester are present in the reactor contents following the 4 hour reaction period. Additionally, substantially all the carboxylic acid was converted to ester over the 4 hr reaction period.

Similar results were obtained for esterification with methanol in examples 2-4 summarized in Table 2. High conversion (86%, 97%, and 98%) to the methyl ester was achieved without esterification catalyst in the 4 hr reaction period.

### Example 5

A 300 mL Parr autoclave reactor was charged with 1.25 g of 3-methylbiphenyl-3′-methylcarboxylate feed compound and 50 g of 1-decanol. No esterification catalyst was added. The reactor was mounted with a condenser that was set to 2° C. The reactor was purged with nitrogen gas. After a pressure test to ensure seal, stirring was started at 600 rpm. The reactor was heated to 250° C. The reactor was pressurized and maintained via pressure regulator at 600 psig. Stirring was continued for 4 hours. Subsequently, heat was removed and pressure slowly reduced. Once reactor had returned to near ambient temperature, the reactor contents were collected and analyzed by gas chromatograph (GC). The decyl ester yield of 3-methylbiphenyl-3′-decylcarboxylate was 10%.

### Example 6

A charge of 10 g (44.25 mmol) of methylbiphenylcarboxylic acid methyl ester isomer mixture was added to a 100 mL round bottom 2-neck flask with a septum cap. The isomer mixture included 3-methylbiphenyl-3-carboxylic acid methyl ester, 4-methylbiphenyl-3-carboxylic acid methyl ester, 3-methylbiphenyl-4-carboxylic acid methyl ester, and 4-methylbiphenyl-4-carboxylic acid methyl ester isomers. The flask and contents were purged with N₂ by cycling between adding N₂ and evacuating for 3-4 cycles. Exxal 10™ alcohol, a commercially available C₁₀ containing alcohol from ExxonMobil, was added to the flask under nitrogen in an amount of 20.8 mL (17.5 g, 110.6 mmol or 2.5 times as many mols of methyl ester) with a magnetic stirbar. A low bubble flow of nitrogen (N₂) was introduced. The temperature of the vial contents was raised to 80° C. with magnetic stirring. N₂ bubbling continued at 80° C. for 2-3 hours to degas. After degassing, the temperature of flask contents was increased to 160° C. TIOT catalyst (Titanium(IV) 2-ethylhexyloxide, CAS 1070-10-6) was added via syringe in an amount of 0.11 mL (100 mg, 0.177 mmol, 0.4 mol %). After addition of TIOT catalyst, the temperature of the flask contents was increased to 190° C. Transesterification was allowed to progress at this temperature with continued N₂ flow and magnetic stirring. The transesterification reaction progress was monitored by GC analysis of periodic samples (i.e., the disappearance of methyl ester and the appearance of C₁₀ ester was monitored).

When conversion to C₁₀ ester reached >99 mol % as measured by GC (1 to 2 hours), the temperature was decreased to 90° C. An amount of 0.56 mL of 5% Na₂CO₃ aq. solution (contains 0.028 g of Na₂CO₃ or 1.5 equivalents Na₂CO₃ to TIOT) was added and stirring was continued at 90° C. for 0.5-1 hour to quench. Subsequently, a small amount (about 25 mg) of charcoal was added and stirring was continued at 90° C. for an additional 0.5-1 hour.

Nitrogen flow and magnetic stirring were stopped. The flask contents (i.e., the third mixture) was filtered through a celite layer (about 50 mg) into another round bottom flask. Methyl alcohol and excess C₁₀ alcohol were removed by Kugelrohr distillation or rotaevap at 120° C. and <1 mm Hg pressure to yield aromatic esters of Exxal 10™ alcohols (i.e., C₁₀ alcohols).

The meanings of terms used herein shall take their ordinary meaning in the art; reference shall be taken, in particular, to Handbook of Petroleum Refining Processes, Third Edition, Robert A. Meyers, Editor, McGraw-Hill (2004). In addition, all patents and patent applications (including priority documents), test procedures (such as ASTM methods), and other documents cited herein are fully incorporated by reference to the extent such disclosure is not inconsistent with this disclosure and for all jurisdictions in which such incorporation is permitted. Also, when numerical lower limits and numerical upper limits are listed herein, ranges from any lower limit to any upper limit are contemplated. Note further that Trade Names used herein are indicated by a™ symbol, indicating that the names may be protected by certain trademark rights, e.g., they may be registered trademarks in various jurisdictions.

The disclosure has been described above with reference to numerous embodiments and specific examples. Many variations will suggest themselves to those skilled in this art in light of the above detailed description. All such obvious variations are within the full intended scope of the appended claims.

1. A method for producing aromatic esters, comprising:
i) providing one or more feed compound(s) of the following Formulas 1(a) and/or 1(b): 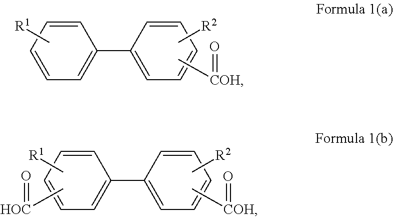
where R¹ is a hydrogen or an alkyl and R² is a hydrogen or an alkyl;
ii) esterifying the feed compound(s) without esterification catalyst with one or more C₁ to C₁₄ alcohols at a temperature from 100° C. to 350° C. and a pressure ≥100 psig to form aromatic esters of the following Formulas 3(a) and/or 3(b): 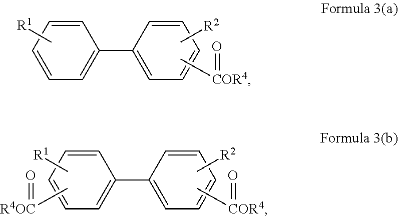
where R¹ and R² are as previously defined and R⁴ is an alkyl residual of the C₁ to C₁₄ alcohol(s).

2. The method of claim 1, where the esterification is at a pressure of ≥250 psig.

3. The method of claim 1, where the esterification is at a pressure of ≥600 psig.

4. The method of claim 1, where the R¹ and/or R² of the feed compound(s) is a methyl group.

5. The method of claim 1, where the feed compounds are one or more isomers of methylbiphenylcarboxylic acid and/or one or more isomers of biphenyldicarboxylic acid.

6. The method of claim 1, where the esterification is at a temperature ≥190° C.

7. The method of claim 1, where R⁴ is an alkyl residual of C₄ to C₁₄ alcohols.

8. The method of claim 1, where the aromatic esters are methyl biphenyl carboxylic acid esters of C₄ to C₁₄ alcohols.

9. The method of claim 1, where the aromatic esters are methyl biphenyl carboxylic acid esters of C₄ to C₁₄ OXO-alcohols.

10. A method for producing aromatic esters, comprising:
i) providing one or more feed compound(s) and one or more impurities, the feed compound(s) having the following Formulas 1(a) and/or 1(b): 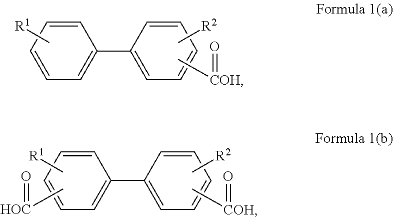
where R¹ is a hydrogen or an alkyl, R² is a hydrogen or an alkyl, the impurities comprising one or more of aldehydes, acetates, aldehyde-acids, acetate-acids, and color body impurity precursors;
ii) esterifying the feed compound(s) without esterification catalyst with methyl or ethyl alcohol at a temperature from 100° C. to 350° C. and a pressure ≥100 psig to form methyl or ethyl esters of the following Formulas 2(a) and/or 2(b): 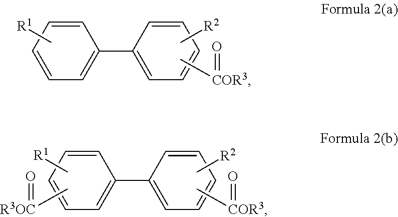
where R¹ and R² are as previously defined and R³ is an alkyl residual of the methyl or ethyl alcohol;
iii) separating the methyl or ethyl esters from substantially all the impurities to form purified methyl or ethyl esters;
iv) transesterifying the purified methyl or ethyl esters with or without esterification catalyst with C₄ to C₁₄ alcohol at a temperature from 100° C. to 350° C. and a pressure at or above ambient pressure to form aromatic esters of the following Formulas 3(a) and/or 3(b): 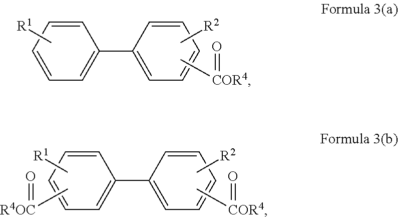
where R¹ and R² are as previously defined and R⁴ is an alkyl residual of C₄ to C₁₄ alcohol.

11. The method of claim 10, where the esterification and/or transesterification is at a pressure of ≥250 psig.

12. The method of claim 10, where the esterification and/or transesterification is at a pressure of ≥600 psig.

13. The method of claim 10, where the feed compounds are one or more isomers of methylbiphenylcarboxylic acid and/or one or more isomers of biphenyldicarboxylic acid.

14. The method of claim 10, where the aromatic esters are methyl biphenyl carboxylic acid esters of C₄ to C₁₄ alcohols.

15. The method of claim 10, where the aromatic esters are methyl biphenyl carboxylic acid esters of C₄ to C₁₄ OXO-alcohols.

16. The method of claim 10, where the feed compounds are esterified with methyl alcohol.

17. The method of claim 10, where the esterification and/or transesterification is at a temperature ≥190° C.

18. A method for producing aromatic polyesters, comprising:
i) providing one or more feed compound(s) and one or more impurities, the feed compound(s) having the following Formula 1(b): 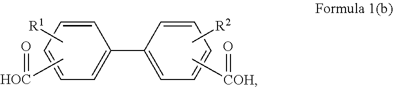
where R¹ is a hydrogen or an alkyl, R² is a hydrogen or an alkyl, the impurities comprising one or more of aldehydes, acetates, aldehyde-acids, acetate-acids, and color body impurity precursors;
ii) esterifying the feed compound(s) without esterification catalyst with methyl or ethyl alcohol at a temperature from 100° C. to 350° C. and a pressure ≥100 psig to form methyl or ethyl esters of the following Formula 2(b): 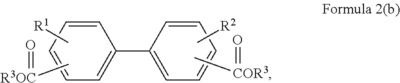
where R¹ and R² are as previously defined and R³ is an alkyl residual of the methyl or ethyl alcohol;
iii) separating the methyl or ethyl esters from substantially all the impurities to form purified methyl or ethyl esters; and
iv) transesterifying the purified methyl or ethyl esters with or without esterification catalyst with one or more diols and, optionally, dimethyl terephthalate at a temperature from 100° C. to 350° C. and a pressure at or above ambient pressure to form polyesters.

19. The method of claim 18, where R¹ and R² are hydrogen.

20. The method of claim 18, where the esterification is at a pressure of ≥250 psig.

21. The method of claim 18, where the esterification is at a pressure of ≥600 psig.

22. The method of claim 18, where the esterification is at a temperature ≥190° C.

23. The method of claim 18, where the diols comprise one or more of the following formulas:

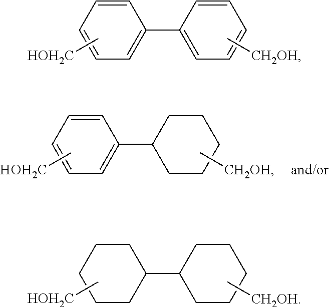

24. The method of claim 18, where the diols comprise a mixture of at least 50 wt % of one or more compounds having the formulas: 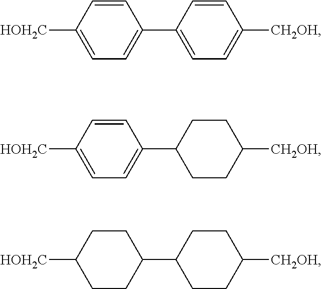 [SMILES: OCc1ccc(-c2ccc(CO)cc2)cc1 ; OCc1ccc(C2CCC(CO)CC2)cc1 ; OCC1CCC(C2CCC(CO)CC2)CC1]
and at least 1 wt % of one or more compounds having the formulas: 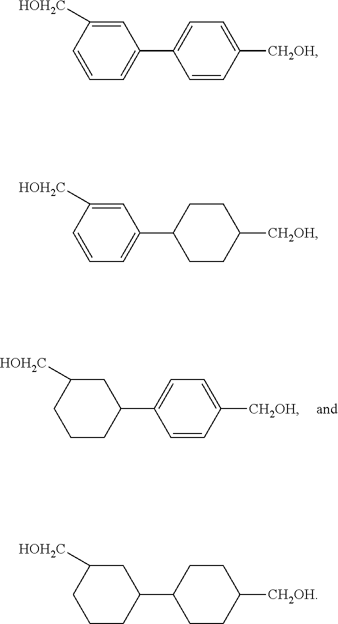 [SMILES: OCc1ccc(-c2cccc(CO)c2)cc1 ; OCc1cccc(C2CCC(CO)CC2)c1 ; OCc1ccc(C2CCCC(CO)C2)cc1 ; "and" ; OCC1CCC(C2CCCC(CO)C2)CC1]
